(19) United States
(12) Patent Application Publication
(10) Pub. No.: US 2022/0015939 A1
(43) Pub. Date: Jan. 20, 2022

(54) ANTI-SNORING PILLOW

(71) Applicant: Alper YENIGÜN, Istanbul (TR)

(72) Inventor: Alper YENIGÜN, Istanbul (TR)

(21) Appl. No.: 17/297,895

(22) PCT Filed: May 8, 2019
(86) PCT No.: PCT/TR2019/050312

§ 371 Date: May 27, 2021

(30) Foreign Application Priority Data
Nov. 27, 2018 (TR) 2018/17923

(51) Int. Cl. A61F 5/56; A47G 9/10

(52) U.S. Cl. CPC A61F 5/56; A47G 2009/1018; A47G 9/1054

(57) ABSTRACT

An anti-snoring pillow that aims to prevent simple snoring and sleep apnea that occurs when lying on back and/or sleeping with lying faces downwards, has a face cavity opened on the surface of the pillow body, allowing the pillow to remain stable and the people to breathe while lying on the face, and wherein the face cavity has a face pad in the pillow body which distributes the weight of the head to the parts touching the face when lying face downwards and takes the shape of the face.

### TECHNICAL FIELD

The present disclosure, in general, aims to prevent simple snoring and sleep apnea that occurs when lying on back.

In particular, the present disclosure relates to a sleeping pillow intended to prevent simple snoring and sleep apnea which is caused by the closure of the airway due to the soft palate and tongue root falling behind when lying in the supine position and/or during sleep by the way of weltering.

### PRIOR ART

In present, sleep problem have a significant effect on the quality of life of individuals. These problems are defined as sleep apnea, snoring and respiratory arrest in sleeping. Sleep, which constitutes an average of one-third of human life, is of great importance for a healthy life. While poor sleep can lead to complaints such as daytime sleepiness and lack of concentration, it also can cause sleep apnea, which can lead to high blood pressure, arrhythmia, paralysis or sudden night deaths. Therefore, for a healthy lifestyle, it is necessary to treat these adverse conditions and/or eliminate them by providing support together with other methods.

In the current technique, first, a detailed otorhinolaryngology examination is performed to the patients who come with complaints of snoring and sleep apnea. If they have a problem relating with their nose, this problem is solved initially by the operations performed on the nose. However, the operations are not enough to eliminate the problems for good.

In the current state of the art, patients with moderate and severe OSAS (obstructive sleep apnea syndrome) who are diagnosed after a sleep test (polysomnography) are directed to chest diseases units to use a CPAP device. The CPAP device is used to pressurize the air in the environment to deliver air to the people using the breathing tube.

While the pressure adjustment and use of this device are under physician supervision, it may cause health problems in possible wrong pressurization adjustments. In addition, because of its structure that surrounds/covers all the head, mouth, and nose area, it negatively affects human psychology (especially in panic attack patients) and offers a structure rather uncomfortable. Also, it does not provide permanent treatment.

In the current state of the art, soft palate surgery is applied to the patients with mild OSAS, if there is a problem concerning soft palate. The surgery is usually performed to shorten the soft palate, to tighten it and to open the airway narrowing behind it to the lateral. Since the success rates of the surgery are low and the use of intra-oral apparatus are difficult, treatment of simple snoring due to soft palate problem cannot be performed effectively.

In addition, in patients with simple snoring and mild OSAS, an intra-oral apparatus placed in the mouth to pull the chin forward is also applied. In these procedures, patients both experience difficulty and pain and are not able to achieve definitive improvement.

In another state of the art, patients are advised to lie to the right or to the left. To prevent these people with simple snoring and mild OSAS from lying on their back, a tennis ball is tied to their back. However, this practice is very primitive, far from the approaches of ergonomics and comfort. People are prevented from lying on their backs, but there is no mechanism that allows them to lie faces downwards comfortably.

In the prior art, the invention in the international patent document CN207574963 relates to a pillow.

This patent document mentions a pillow supporting the head, neck, and shoulder. There is a protrusion in the middle of the pillow that corresponds to the neck cavity. Due to this protrusion, the chin is lifted up, the airway is flattened and effective breathing is provided. However, the said pillow is not suitable for facedown use, because it includes a mandatory structure for lying on back use. As it is known, sleeping in supine position is insufficient to solve the problem of snoring and apnea.

As a result, improvements are made in the anti-snoring pillow, so new structures are needed to eliminate the disadvantages mentioned above and to bring solutions to the existing systems.

### THE PURPOSE OF THE INVENTION

The present invention relates to an anti-snoring pillow that meets the above-mentioned requirements, eliminates all disadvantages and brings some additional advantages.

The main purpose of the invention is to eliminate the snoring and sleep apnea problems that occur in people during sleep.

A further purpose of the invention is to prevent the soft palate and tongue root falling backward due to gravity, especially during sleeping in laying supine position, and to prevent the snoring and sleep apnea problems without the need for surgical operations.

Another purpose of the invention is, especially in patients with simple snoring and mild OSAS complaints, to increase the quality of sleep with the use of the anti-snoring pillow without requiring the use of primitive methods and techics which are hard to use such as intra-oral apparatuses.

Another purpose of the invention is to enable people who have difficulty breathing while lying supine position to allow them to lie faces downwards and breathe comfortably.

In order to accomplish all the advantages above and that will be explained below, the invention is an anti-snoring pillow that aims to prevent simple snoring and sleep apnea that occurs when lying supine position and/or sleeping with lying faces downwards, wherein; comprises a face cavity opened on the surface of the pillow body, allowing the pillow to remain stable and to breathe while lying on the face, and that said face cavity comprises a face pad in said pillow body which distributes the weight of the head to the parts touching the face when lying face downwards and takes the shape of the face.

The structural and characteristic features and all advantages of the invention outlined in the figures below and in the detailed description made by referring these figures will be understood clearly. Therefore, the evaluation should be made considering these figures and detailed explanations.

### BRIEF DESCRIPTION OF THE FIGURES

In order to be able to understand the advantages of the present invention together with the additional elements, it is necessary to evaluate it with the figures explained below.

FIG. 1 is the front view of the anti-snoring pillow.

FIG. 2 is the back view of the anti-snoring pillow.

FIG. 3 is the section view of the anti-snoring pillow.

FIG. 4 is the perspective view of the anti-snoring pillow.

FIG. 5 is a view showing the face downwards use of the anti-snoring pillow.

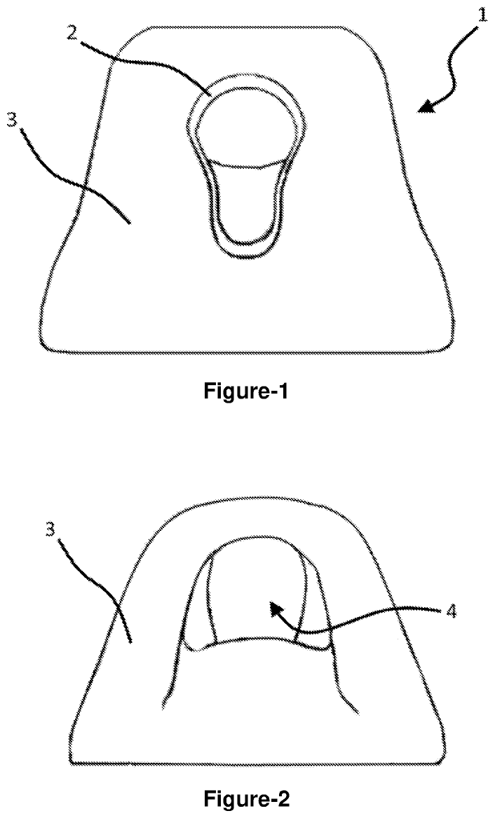

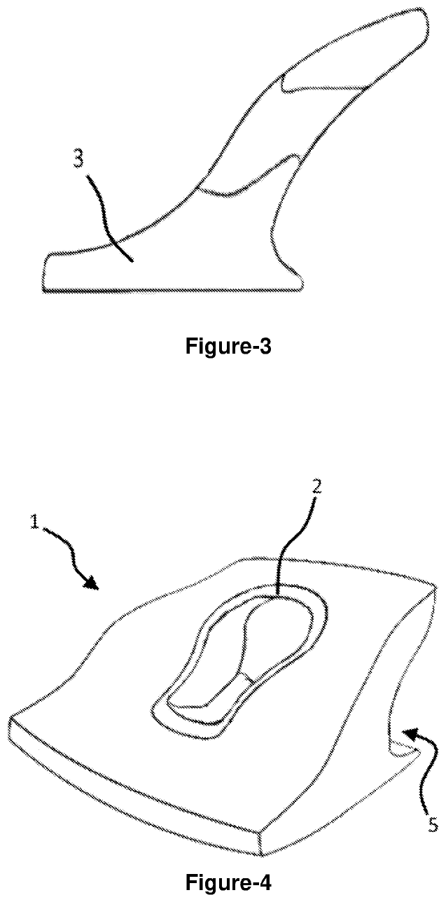

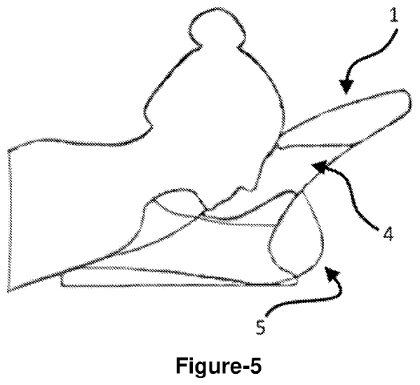

### REFERENCE NUMBERS

1. Pillow
2. Face pad
3. Pillow body
4. Face cavity
5. Arms gap

### DETAILED DESCRIPTION OF THE INVENTION

In this detailed description, preferred structures of the anti-snoring pillow (1) of the invention are explained only for a better understanding of the subject matter and without any restrictive effect.

The pillow (1) of the invention eliminates all the above-mentioned problems, and minimizes sleep apnea and snoring. In the middle of the said pillow (1), there is a face pad (2) having a soft spongy structure. The pillow (1) comprises a face pad (2) that contacts with the face when lying facedown position distributing the weight of the head to the parts where the face is in contact on the pillow and can take the shape of the face. The pillow face pad (2) that is a soft-built and can take the shape of the face made of a viscoelastic sponge material. The visco is a material that is capable of heat formation and does not irritate the skin. The pillow is shaped by the weight of the head of a person lying on the face pad (2) made of said visco-material and with this flexibility, equal pressure is applied to each point around the face.

The parts that do not touch/contact with the face constitutes the pillow body (3). The pillow body (3) is intended to provide the use of pillow (1) in lying positions such as on the back, to right and left. Moreover, with its full structure, it ensures that the pillow (1) remains stable in the supine position, in face downwards. For this reason, it is designed using a more rigid sponge material. Thus, the person can sleep comfortably in the prone position (lying face downwards) without any difficulty in breathing while using the anti-snoring pillow (1).

In the middle part of the said pillow (1), there is a cavity that allows breathing comfortably while lying face downwards. This cavity in the form of facial lines is defined as the face cavity (4) and the face pad (2) surrounds this face cavity. In addition to all these, there is another opening called the arms gap (5) at the back of the pillow (1). The arms gap (5) allows the arms to move forward and allows to sleep in accordance with the spine.

By its structure, the pillow (1) can be designed by using all the materials (sponge, plastic, cotton, fiber, wool, etc.) used in the manufacturing of a pillow (1). It is possible to use various materials as it is designed for individuals to sleep in the prone position, without having difficulty in breathing. High or low-pressure polyurethane insulation foam can be injected into the mold as polyurethane liquid by injection machine and can be densified with heat. When cooled, the pillow body (3) containing the desired hardness sponge structure can be obtained. Mentioned pillow (1) provides the opportunity to sleep in face downwards and allows the airline to stay clear (open). Thus, it prevents snoring and sleep apneas. The pillow (1) achieves to increase the quality of sleep especially in patients with simple snoring and OSAS complaints.

1. A pillow that aims to prevent simple snoring and sleep apnea that occurs when lying and/or sleeping supine position with lying faces downwards, the pillow comprising:
a face cavity opened on the surface of a pillow body, allowing the pillow to remain stable and the people to breathe while lying on the face;
a face pad in said pillow body of face cavity which distributes the weight of the head to the parts touching the face when lying face downwards and can take the shape of the face.

2. A pillow according to claim 1, comprising an arms gap which allows the arms to move forward when sleep by using the pillow and provides lying comfortable by the way of cross around the pillow.

3. A pillow according to claim 1, wherein said face pad is made of a viscoelastic sponge material.

4. A pillow according to claim 1, wherein the pillow body, which allows the pillow to remain stable, is a hard-built sponge made of polyurethane material.
